(19) United States
(12) Patent Application Publication
(10) Pub. No.: US 2023/0058544 A1
(43) Pub. Date: Feb. 23, 2023

(54) METHOD FOR INCREASING EICOSAPENTAENOIC ACID LEVEL IN THE PLASMA OF AN ANIMAL

(71) Applicant: DSM IP Assets B.V., HEERLEN (NL)

(72) Inventor: Shiguang YU, Columbia, MD (US)

(21) Appl. No.: 17/797,652

(22) PCT Filed: Feb. 5, 2021
(86) PCT No.: PCT/US2021/016701

§ 371 Date: Aug. 4, 2022

Related U.S. Application Data
(60) Provisional application No. 62/970,932, filed on Feb. 6, 2020.

(51) Int. Cl. A23K 50/42; A23K 20/158

(52) U.S. Cl. CPC A23K 50/42; A23K 20/158

(57) ABSTRACT

Disclosed herein is a method for increasing the plasma EPA:DHA ratio in a non-human animal by feeding the animal with DHA-rich polyunsaturated fatty acid diet. One example of such DHA-rich polyunsaturated fatty acid diet contains at least 0.6% DHA by weight of the feed. Also disclosed herein is an animal feed composition comprising DHA-rich polyunsaturated fatty acid. One example of such animal feed composition is pet food.

### FIELD OF THE TECHNOLOGY

The present invention relates to a method to increase the eicosapentaenoic acid (“EPA”) level in the plasma of said animals given docosahexaenoic acid (“DHA”) orally at a fixed ratio of DHA to EPA.

### CROSS REFERENCE TO RELATED APPLICATIONS

This application is an International Application claiming priority to U.S. Provisional Application No. 62/970,932, filed Feb. 6, 2020, the entire contents of which are hereby incorporated by reference in their entirety.

### BACKGROUND OF THE INVENTION

The nutritional value of polyunsaturated fatty acids (PUFAs) such as omega-3 fatty acids are well known in the art. PUFAs are biologically important molecules which affect cellular physiology due to their presence in cell membrane. PUFAS also regulate the production of gene expression of biologically active compounds and serve as biosynthetic substrates. For example, DHA accounts for approximately 15%-20% of lipid in the animal's brain and 30%-60% of lipids in the retina (Jean-Pascal Berge & Gilles Barnathan, Fatty Acids from Lipids of Marine Organisms: Molecular Biodiversity, Roles as Biomarkers, Biologically Active Compounds, and Economical Aspects, in Marine Biotechnology I 49,T. Scheper, ed., 2005). Evidence from multiple studies has shown that intake of recommended amounts of EPA in the form of dietary supplements reduces inflammation (Lau C S, Morley K D, Belch J J. Br. J. Rheumatol. 1993; 32(11):982-989; Kremer J M, Lawrence D A, Petrillo G F, et al. Arthritis Rheum. 1995; 38(8):1107-1114). Because omega-3 fatty acids cannot be synthesized de novo by land animals, these fatty acids must be obtained from nutritional sources.

Polyunsaturated fatty acids are synthesized by microbes such as microalgae and fungi. Fish acquires polyunsaturated fatty acids by feeding on such microbes. Commercially, polyunsaturated fatty acids are obtained by extraction from fish, or by harvesting from microalgae or fungi via fermentation. Polyunsaturated fatty acids extracted from certain fish species, which is often called “fish oil”, has a high % of EPA and a relatively low % of DHA. Polyunsaturated fatty acids extracted from microalgae, which is often called “algal oil”, have various amount of EPA and DHA. For example, some algal oils have a high % of DHA and a relatively low % of EPA. Some other algal oils have a high % of DHA and almost no EPA.

It is known that when EPA-related health benefits are desired for an animal, direct intake of high amount of EPA can significantly increase the concentration of EPA in the plasma of such animal. However, there are circumstances when direct intake of EPA is infeasible. For example, the commercial supply of fish oil may be low from time to time due to climate reasons. In other circumstances, the use of fish oil in premier pet food may be undesirable because of the existence of pollutants such as mercury.

Thus, there is a need for increasing EPA concentration in animal's plasma by supplying one or more type of polyunsaturated fatty acids which is not EPA.

### BRIEF SUMMARY OF THE INVENTION

The invention is directed to a method for increasing the plasma EPA:DHA ratio in a non-human animal, wherein said non-human animal is fed with a feed comprising at least 0.6% DHA by weight. In one embodiment, the feed comprises at least 0.68% DHA by weight. In another embodiment, said feed further comprises 0.21% or less EPA by weight. In another embodiment, said the EPA:DHA ratio of said feed is 0.39:1 or less. In another embodiment, the above plasma EPA:DHA ratio is increased at least one fold when comparing to the plasma EPA:DHA ratio of a control group of animals which is fed with a feed comprising 0.1% or less DHA. In another embodiment, the above plasma EPA:DHA ratio is increased at least one fold, at least two folds, at least three folds, at least four folds, or at least five folds, when comparing to the plasma EPA:DHA ratio of a control group of animals which is fed with a feed containing no polyunsaturated fatty acid.

In some embodiments, the above non-human animal is a pet animal, such as a dog, a cat, or a horse.

In some embodiments, the above non-human animal is a monogastric animal, a ruminant animal, a zoological animal, a work animal, or a livestock.

The invention is also directed to a method for attaining a customized level of plasma EPA:DHA ratio in a non-human animal by feeding said non-human animal with DHA-rich feed, wherein said method comprises the steps of: 1) collecting three or more plasma EPA:DHA ratio in said non-human animal and their corresponding level of DHA in the feed; 2) calculating the correlation coefficient index based on the data in step 1); 3) determine the customized level of DHA in the feed using the correlation coefficient index in step 2); and 4) feed non-human animal with a feed comprising the level of DHA determined in step 3).

In one embodiment, said non-human animal is a pet animal, such as a dog, a cat, or a horse.

In other embodiments, said non-human animal is a monogastric animal, a ruminant animal, a zoological animal, a work animal, or a livestock.

The invention is also directed to a feed composition for a non-human animal, which is used for increasing the plasma EPA:DHA ratio in said non-human animal, wherein the feed comprises at least 0.6% DHA by weight.

In one embodiment, said non-human animal is a pet animal, such as a dog, a cat, or a horse.

In other embodiments, said non-human animal is a monogastric animal, a ruminant animal, a zoological animal, a work animal, or a livestock.

### BRIEF SUMMARY OF DRAWINGS

FIG. 1 is a graph illustrating the increase of EPA:DHA ratio in cats' plasma when the cats were fed with food containing different amounts of algal oils with a fixed EPA:DHA ratio.

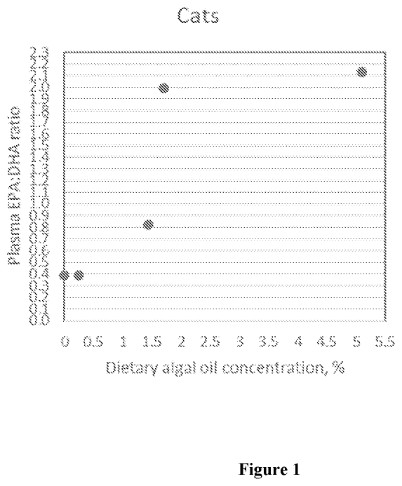

FIG. 2 is a graph illustrating the increase of EPA:DHA ratio in dogs' plasma when the dogs were fed with food containing different amounts of algal oils with a fixed EPA:DHA ratio.

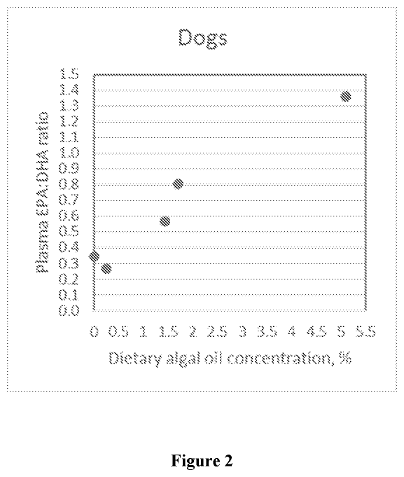

### DETAILED DESCRIPTION OF THE INVENTION

The features and advantages of the invention may be more readily understood by those of ordinary skill in the art upon reading the following detailed description. It is to be appreciated that certain features of the invention that are, for clarity reasons, described above and below in the context of separate embodiments, may also be combined so as to form sub-combinations thereof.

Embodiments identified herein as exemplary are intended to be illustrative and not limiting.

It is an objective of the present invention to provide a DHA-rich PUFA oil with a fixed EPA:DHA ratio which can significantly increase the plasma EPA:DHA ratio in a non-human animal.

The present invention accomplished the above-identified objective by giving the animal a threshold or higher level of DHA. The inventors have discovered unexpectedly that by providing a DHA-rich oil with a fixed ratio of EPA:DHA to an animal, the plasma EPA:DHA ratio of said animal increases as the level of DHA in the ingested DHA-rich oil increases. Surprisingly, the EPA:DHA ratio in the plasma of the animal did not conform to the EPA:DHA ratio of the DHA-rich oil ingested. Instead, the EPA:DHA ratio in the plasma of the animal after it ingests the DHA-rich oil becomes significantly higher than the EPA:DHA ratio of the oil that is fed to the animal. In other words, the plasma EPA concentration increases significantly more than the increase of the plasma DHA concentration in an animal, even when the EPA:DHA ratio of the oil which is ingested by the animal is fixed. Furthermore, the inventors have discovered that the oversized increase of EPA concentration in plasma does not occur at lower amount of DHA-rich oil intake. Instead, it occurs only after a minimal amount of DHA-rich oil has been ingested. Based on the above observations, the inventors have conceived a method to increase the plasma EPA:DHA ratio in a non-human animal by providing a DHA-rich oil containing a threshold level of DHA. In one embodiment, said DHA-rich oil has a fixed % of DHA and EPA and thus a fixed EPA:DHA ratio.

The present invention thus discloses a minimal level of DHA required to significantly increase the plasma EPA concentration in the non-human animal.

One embodiment of the present invention is directed to a method for increasing the plasma EPA:DHA ratio in a domestic animal, wherein said domestic animal is fed with a feed comprising at least 0.6% DHA by weight. In another embodiment, the feed may comprise at least 0.68% DHA by weight. In other embodiments, the feed may comprise at least 0.7%, at least 0.8%, at least 0.9%, at least 1.0%, at least 1.1%, at least 1.2%, at least 1.3%, at least 1.4%, at least 1.5%, at least 1.6%, at least 1.7%, at least 1.8%, at least 1.9%, or at least 2.0% DHA by weight.

As discussed above, the inventors have discovered that by supplying a threshold level of DHA in the feed to a non-human animal, the EPA:DHA ratio in the plasma of the animal can become significantly higher than the EPA:DHA ratio of the DHA-rich oil supplied in the diet. This observation suggests that EPA in the diet may not be the sole source for the animal to absorb into its plasma. It is the inventors' hypothesis that some of the DHA in the diet has been converted into EPA and thus causes the oversized increase of EPA in the plasma. However, this is just one of the many possible hypotheses to explain the observation made by the inventors. This invention should by no means be bound by this theory.

In the context of the present invention, the phrase “increasing the plasma EPA:DHA ratio” refers to the increase of plasma EPA:DHA ratio when comparing a test group of animals which is given the DHA-rich oil feed to a control group of animals which is given the same feed except that the feed contains little of or no such DHA-rich oil. In one embodiment, the feed supplied to the control group animal comprises less than 0.1% DHA. In another embodiment, the feed supplied to the control group animal comprises no DHA. In another embodiment, the feed supplied to the control group animal comprises no PUFA oil at all. The plasma EPA:DHA ratio in the control group of animals has an intrinsic plasma EPA:DHA ratio. In one embodiment, this intrinsic plasma EPA:DHA ratio in the control animal is at or around 0.39:1.

In another embodiment of the invention, the feed provided to the test group of animals comprises not only DHA but also other polyunsaturated fatty acids, such as EPA, arachidonic acid (ARA), docosapentaenoic acid (DPA) n-3, DPAn-6, myristic acid, palmitic acid, and palmitoleic acid. In one embodiment, the feed provided to the test group of animals comprises only a small amount of EPA. In one embodiment, the amount of EPA in the feed provided to the test group of animals is less than 0.21% by weight. In another embodiment, the feed provided to the test group of animals contains no EPA.

The method disclosed in the present invention can significantly increase the plasma EPA:DHA ratio of a non-human animal when it is fed with a feed comprising at least 0.6% DHA by weight. Such increase is observed when the level of plasma EPA:DHA ratio is compared to the level of plasma EPA:DHA ratio of a control group of animals. In one embodiment, the control group of animals is fed with a feed containing no PUFA oil. In another embodiment, the test group of animals is fed with a feed containing no DHA. In another embodiment, the control group of animals is fed with a feed containing less than 0.1% DHA. In another embodiment, the control group of animals is fed with a feed containing less than 0.1% DHA and EPA. In another embodiment, the control group of animals is fed with a feed containing less than 0.1% PUFA. In one embodiment, such increase of EPA:DHA ratio is one-fold. In another embodiment, the increase of EPA:DHA ratio is two-fold. In another embodiment, the increase of EPA:DHA ratio is three-fold. In another embodiment, the increase of EPA:DHA ratio is four-fold. In another embodiment, the increase of EPA:DHA ratio is five-fold. The word “fold” or “one fold” means a 100% increase over the starting value. For example, if the average plasma EPA:DHA ratio of the control group of animals is 0.4, one fold increase of the plasma EPA:DHA ratio is 0.8.

The present invention also makes it possible to increase the plasma EPA:DHA ratio of a non-human animal to a target range or value. For example, if it has been found that a plasma EPA:DHA ratio of 2:1 is proven to be able to significantly reduce the occurrence of inflammation in cats, such EPA:DHA ratio can be reached by supplying the cats with a feed containing a DHA-rich oil with more than 0.68% DHA by weight of the feed. It is discovered by the inventors that by increasing the DHA level in feed, the plasma EPA:DHA ratio of such animal can increase significantly. The degree of increase of the EPA:DHA ratio may vary from animals to animals, as demonstrated in Example 1. However, the correlation coefficient index for any given animal can be determined by routine experimentation. Thus, the present invention is directed to a method for raising the plasma EPA:DHA ratio in a non-human animal to a set level, wherein said non-human animal is fed with a feed comprising customized level of DHA with a fixed EPA:DHA ratio. The method comprises the steps of: 1) collecting three or more plasma EPA:DHA ratio in said non-human animal and their corresponding level of DHA in the feed; 2) calculating the correlation coefficient index based on the data in step 1); 3) determine the customized level of DHA in the feed using the correlation coefficient index in step 2); and 4) feed non-human animal with a feed comprising the level of DHA determined in step 3). In one embodiment, the three or more plasma EPA:DHA ratios were measured in an animal when feed with three or more different level of DHA are fed to the animal. In some embodiments, at least three, at least four, at least five, at least seven plasma EPA:DHA ratios in the non-human animal and their corresponding level of DHA in the feed are collected or measured. For example, five plasma EPA:DHA ratios in cats and dogs and their corresponding level of DHA in the feed were measured in Example 1 of this application.

The present invention is directed to feed composition comprising a DHA-rich PUFA oil, wherein the feed helps to increase the plasma EPA:DHA ratio in a non-human animal. The source of such DHA rich PUFA oil may derive from microorganisms such as microalgae, fungi, bacteria, yeast, or protist, or from marine animal such as fish. Regardless of the source of the PUFA oil, it is preferred that such PUFA oil is rich in DHA. The term “rich in DHA” means that the % DHA in the PUFA oil is higher than the % EPA, % DPAn-3, % DPAn-6, and % ARA in the same oil. In some embodiments, the % DHA in the PUFA oil is higher than 20% by weight of the PUFA oil, higher than 30% by weight of the PUFA oil, higher than 40% by weight of the PUFA oil, and higher than 50% by weight of the PUFA oil.

The present invention is directed to an animal feed composition comprising any of the PUFA oils of the invention for a non-human animal. In one embodiment, the animal feed composition is pet food. In one embodiment, the animal feed composition is animal feed.

An “animal” includes domestic animals; farm animals; zoo animals; sport animals; breeding stock; racing animals; show animals; heirloom animals; rare or endangered animals; companion animals; pet animals such as dogs, cats, guinea pigs, rabbits, rats, mice, or horses; primates such as monkeys, apes, orangutans, baboons, gibbons, and chimpanzees; canids such as dogs and wolves; felids such as cats, lions, and tigers; equids such as horses, donkeys, and zebras; food animals such as cows, cattle, pigs, and sheep; ungulates such as deer and giraffes; or rodents such as mice, rats, hamsters and guinea pigs; monogastric animals such as cats, dogs, horses, or pigs, or ruminants such as cows and sheep, and so on. An animal feed includes, but is not limited to, a domestic animal feed including pet feed, a zoological animal feed, a work animal feed, a livestock feed, and combinations thereof. In a preferred embodiment, the method of the present application is applied to a monogastric animal. However, the method of the present application can also be applied to any non-monogastric animals, for example, a ruminant animal.

It is known that there are variety of feed receipts for any given animal species, and there are also variety of feeding regimes. Some of these feed receipts and feeding regimes are significantly different from others. However, the effect of the present invention is determined by the amount of polyunsaturated fatty acids oil used and especially the amount of DHA used in the feed, and is not impacted by the variation in feed compositions or in feeding regimes.

### EXAMPLES

### Example 1

Adult cats (>1 year) and adult dogs (>1 year) were given generic dry cat or dog food which is supplemented with an algal oil in the amount of 0%, 0.25%, 1.44%, 1.7%, or 5.1% by weight of the cat food or dog food for 4 to 5 weeks. The algal oil used in the studies contains 12.5% EPA and 40% DHA with an EPA to DHA ratio of 0.31. A control group of cats and dogs are fed with a control diet in which the 0.25%, 1.44%, 1.7%, or 5.1% of the algal oil is replaced by poultry fat. The dry pet food without the algal oil supplementation (0%) was nutritionally complete and balanced according to the Association of American Feed Control Officials (AAFCO) nutrient profiles. This pet food without the algal oil supplementation (0%) was the control diet. As a result, all test diets had EPA to DHA ratio of 0.31.

All cats or dogs were fed with their control diets for at least 4 weeks before they were given their designated test diets. The control group had 20 cats or dogs. The test group each had 10 cats or dogs. Blood samples were taken at the end of the studies. Plasma EPA and DHA concentrations were analysed and EPA to DHA ratio in the plasma was calculated.

The plasma EPA to DHA ratio in cats and dogs are summarized in Table 1. The plasma EPA to DHA ratio was not affected by dietary algal oil concentrations up to 1.44% (p>0.05), although the ratio increased numerically at the higher dietary algal oil concentration (1.44%). However, further increase of the amount of algal oil included in the feed (1.7% and 5.1%) significantly increased the plasma EPA to DHA ratio (p<0.05) in both cats and dogs.

When the dietary algal oil concentration is 1.7%, the dietary EPA concentration is 0.21%, and the dietary DHA concentration is 0.68%.

TABLE 1

| | Cat Plasma | | | |
|---|---|---|---|---|
| Algal Oil, | EPA:DHA | | Dog Plasma | |
| % | ratio | P Values* | EPA:DHA ratio | P Values* |
| 0 (Control) | 0.39 | | 0.34 | |
| 0.25 | 0.39 | 0.996 | 0.27 | 0.060 |
| 1.44 | 0.82 | 0.057 | 0.57 | 0.099 |
| 1.7 | 1.99 | <0.01 | 0.80 | 0.003 |
| 5.1 | 2.13 | <0.01 | 1.36 | <0.01 |

*Student t-test, each test diet vs. the control diet

1.-27. (canceled)

28. A method for increasing the plasma EPA:DHA ratio in a non-human animal, wherein said non-human animal is fed with a feed comprising at least 0.6% DHA by weight.

29. The method of claim 28, wherein said feed comprises at least 0.68% DHA by weight.

30. The method of claim 29, wherein said feed further comprises 0.21% or less EPA by weight.

31. The method of claim 28, wherein the EPA:DHA ratio of said feed is 0.39:1 or less.

32. The method of claim 28, wherein said plasma EPA:DHA ratio is increased at least one fold when comparing to the plasma EPA:DHA ratio of a control group of animals which is fed with a feed comprising 0.1% or less DHA.

33. The method of claim 28, wherein said non-human animal is a pet animal.

34. The method of claim 33, wherein said pet animal is a dog, a cat, or a horse.

35. The method of any one of claim 28, wherein said non-human animal is a monogastric animal.

36. The method of any one of claim 28, wherein said non-human animal is a ruminant animal.

37. A method for attaining a customized level of plasma EPA:DHA ratio in a non-human animal by feeding said non-human animal with DHA-rich feed, wherein said method comprises the steps of:
1) collecting three or more plasma EPA:DHA ratio in said non-human animal and their corresponding level of DHA in the feed;
2) calculating the correlation coefficient index based on the data in step 1);
3) determine the customized level of DHA in the feed using the correlation coefficient index in step 2); and
4) feed non-human animal with a feed comprising the level of DHA determined in step 3).

38. The method of claim 37, wherein said non-human animal is a pet animal.

39. The method of claim 38, wherein said pet animal is a dog, a cat, or a horse.

40. The method of claim 37, wherein said non-human animal is a monogastric animal.

41. The method of claim 37, wherein said non-human animal is a ruminant animal.

42. A feed composition for a non-human animal, which is used for increasing the plasma EPA:DHA ratio in said non-human animal, wherein the feed comprises at least 0.6% DHA by
